SEQUENCE LISTING (8 sequences)

SEQ ID NO: 1 - protein, 309 aa, Homo sapiens
  human uncoupling protein 2 (hUCP2) Chen (Tularik)
MVGFKATDVPPTATVKFLGAGTAACIADLITFPLDTAKVRLQIQGESQGPVRATASAQYRGVMGTILTMVRTEGPRSLYNGLVAGLQRQMSFASVRIGLYDSVKQFYTKGSEHASIGSRLLAGSTTGALAVAVAQPTDVVKVRFQAQARAGGGRRYQSTVNAYKTIAREEGFRGLWKGTSPNVARNAIVNCAELVTYDLIKDALLKANLMTDDLPCHFTSAFGAGFCTTVIASPVDVVKTRYMNSALGQYSSAGHCALTMLQKEGPRAFYKGFMPSFLRLGSWNVVMFVTYEQLKRALMAACTSREAPF

SEQ ID NO: 2 - DNA, 930 bp, Homo sapiens
  CDS (1)..(930): human uncoupling protein 2 (hUCP2) Chen (Tularik)
atggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcacttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctga

SEQ ID NO: 3 - DNA, 34 bp, Artificial Sequence
  Description of Artificial SequenceU1F primer
atcaagcttatggttgggttcaaggccacagatg

SEQ ID NO: 4 - DNA, 33 bp, Artificial Sequence
  Description of Artificial SequenceU8R primer
atcggatcctcagaagggagcctctcgggaagc

SEQ ID NO: 5 - DNA, 930 bp, Homo sapiens
  CDS (1)..(930): human ucoupling protein 2 (hUCP2) Tartaglia et al.
atggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagtcagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcacttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctga

SEQ ID NO: 6 - protein, 309 aa, Homo sapiens [sequence omitted]

SEQ ID NO: 7 - DNA, 930 bp, Homo sapiens
  CDS (1)..(930): human uncoupling protein 2 (hUCP2) Fleury et al.
atggttgggttcaaggccacagatgtgccccctactgccactgtgaagtttcttggggctggcacagctgcctgcatcgcagatctcatcacctttcctctggatactgctaaagtccggttacagatccaaggagaaagtcaggggccagtgcgcgctacagccagcgcccagtaccgcggtgtgatgggcaccattctgaccatggtgcgtactgagggcccccgaagcctctacaatgggctggttgccggcctgcagcgccaaatgagctttgcctctgtccgcatcggcctgtatgattctgtcaaacagttctacaccaagggctctgagcatgccagcattgggagccgcctcctagcaggcagcaccacaggtgccctggctgtggctgtggcccagcccacggatgtggtaaaggtccgattccaagctcaggcccgggctggaggtggtcggagataccaaagcaccgtcaatgcctacaagaccattgcccgagaggaagggttccggggcctctggaaagggacctctcccaatgttgctcgtaatgccattgtcaactgtgctgagctggtgacctatgacctcatcaaggatgccctcctgaaagccaacctcatgacagatgacctcccttgccacttcatttctgcctttggggcaggcttctgcaccactgtcatcgcctcccctgtagacgtggtcaagacgagatacatgaactctgccctgggccagtacagtagcgctggccactgtgcccttaccatgctccagaaggaggggccccgagccttctacaaagggttcatgccctcctttctccgcttgggttcctggaacgtggtgatgttcgtcacctatgagcagctgaaacgagccctcatggctgcctgcacttcccgagaggctcccttctga

SEQ ID NO: 8 - protein, 309 aa, Homo sapiens [sequence omitted]

(19) United States
(12) Patent Application Publication
(10) Pub. No.: US 2002/0127600 A1
(43) Pub. Date: Sep. 12, 2002

(54) Human uncoupling protein-2 (hCP2): compositions and methods of use

(76) Inventors: Jin-Long Chen, Millbrae, CA (US); M. Catherine Amaral, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/884,814

(22) Filed: Jun. 18, 2001

Related U.S. Application Data
(63) Continuation of application No. 09/124,293, filed on Jul. 29, 1998, now abandoned.

(51) Int. Cl. C12N 5/02; C12N 5/00; C12P 21/06; C07H 21/04; A61K 38/00; G01N 33/53; C12Q 1/68

(52) U.S. Cl. 435/7.1; 536/23.5; 435/325; 435/6; 514/12

(57) ABSTRACT

The present invention relates generally to compositions and methods for the treatment of body weight disorders including, but not limited to, obesity. More specifically, the present invention relates to nucleic acids encoding a human UCP2 polypeptide; a human UCP2 polypeptides encoded by such nucleic acids; recombinant nucleic acid molecules containing nucleic acids encoding a human UCP2 polypeptide; cells containing such recombinant nucleic acid molecules; a method for producing human UCP2 polypeptides; and methods for detecting modulators of UCP2 gene expression and UCP2 polypeptide expression.

### FIELD OF THE INVENTION

The present invention relates generally to compositions and methods for the treatment of body weight disorders including, but not limited to, obesity. More specifically, the present invention relates to nucleic acids encoding a human UCP2 polypeptide; a human UCP2 polypeptides encoded by such nucleic acids; recombinant nucleic acid molecules containing nucleic acids encoding a human UCP2 polypeptide; cells containing such recombinant nucleic acid molecules; a method for producing UCP2 polypeptides; and methods for detecting modulators of UCP2 gene expression and UCP2 polypeptide expression.

### BACKGROUND OF THE INVENTION

Body weight disorders, including eating disorders, represent major health problems in all industrialized countries. Obesity has reached epidemic proportions in the United States and is threatening to become a global epidemic. According to the classification scheme recently established by the World Health Organization, 54% of U.S. adults are overweight and 22% are obese (see, World Health Organization, Obesity: Preventing and Managing the Global Epidemic (World health Organization, Geneva, 1998). The prevalence of overweight people has risen dramatically over the past two decades and, if this trend persists, the entire U.S. adult population will be overweight within a few generations. Obesity represents a serious threat to health because it increases the risk of developing many chronic diseases, such as diabetes and cardiovascular diseases. Other body weight disorders, such as anorexia nervosa and bulimia nervosa, which together affect approximately 0.2% of the female population of the western world, also pose serious health threats. It has been found that body weight disorders, such as anorexia nervosa and cachexia (i.e., wasting) are also prominent features of other diseases such as cancer, cystic fibrosis and AIDS.

Just about everybody who has struggled to shed and keep off pounds has envied those lucky few who can apparently eat whatever they want and never gain a pound. Metabolism—the way an individual break's down food and uses it for energy—may make at least part of the difference. Some people simply have lower metabolic rates and, thus, a greater tendency to gain weight than others. It is only recently that researchers are beginning to get a handle on what accounts for those differences. More particularly, researchers have now identified what appear to be the first human “uncoupling proteins” (UCPs). Originally discovered decades ago in the special brown fat cells that animals, such as bears, burn up while hibernating, UCPs are so called because they dissociate the reactions that break down food from those that produce the body's chemical energy. In effect, they punch holes in the energy-production pipeline, raising the body's resting metabolic rate.

Because the lost chemical energy is dissipated as heat, UCPs help hibernators and other cold-adapted animals maintain their core body temperatures in frigid weather. But people do not have brown fat, except in small amounts when they are newborns, and it was thought that the UCP proteins did not have much effect on human metabolism. However, recent work now challenges this assumption because it shows that other human tissues, including ordinary fat and muscle, make proteins very similar to the animal UCPs. As such, great efforts are being made to pin down the role of UCP proteins because, if human UCPs do have the predicted function, their discovery could help provide a better understanding of obesity as well as improved treatments for this condition. It is thought that variations in UCP production or activity may be what cause some people to have lower or higher metabolic rates than others and, thus, a greater or lesser tendencies to get fat.

The first uncoupling protein (UCP1) was independently discovered in the mid-1970s by biochemist David Nicholls at the University of Dundee in the U.K. and Daniel Ricquier at the National Center for Scientific Research (CNRS) in Paris. At the time, researchers already knew that hibernating animals, and also cold-adapted rodents, use special fat cells (i.e., the brown adipocytes) to produce body heat. To try to find out more about how these cells work, Ricquier kept lab rats in either cold or warm temperatures and then looked for differences in the proteins made by the brown fat cells. In doing so, it was found that the fat cells of the animals in cold temperatures produced a 32-kilodalton protein that is not made by the animals in warm temperatures.

At about the same time, Nicholls and his team identified the mitochondria, i.e., the tiny kidney-shaped organelles that serve as the cells' powerhouses, as the source of heat released by brown fat. The mitochondria use the energy contained in dietary sugars, fats and other nutrients to drive the synthesis of the high-energy compound adenosine triphosphate (ATP). This process depends on an electrochemical gradient set up across the inner of the two mitochondrial membranes when protons (positively charged hydrogen ions) are pumped out of the interior chamber of the mitochondrion.

By injecting a radioactive compound into fat cells and then measuring its concentration on either side of the mitochondrial membrane, it was shown that the inner membrane of brown fat mitochondria is very permeable to protons. Ultimately, the researchers traced this leak to a protein in the mitochondrial membrane that came to be known as UCP 1. By creating the leak, UCP1 reduces the number of ATPs that can be made from a given amount of food, thereby raising the body's metabolic rate and generating heat. Normally, though, the protein is kept in an inactive state by nucleotides that bind to the protein. Then, when the animal needs extra heat, it activates neurons that release the neurotransmitter norepinephrine at the surfaces of the brown fat cells, and the hormone then sets in motion a chain of events that releases the inhibition.

Humans have a UCP1 gene, but it is active only in their brown fat, which disappears shortly after birth. Still, measurements of the amount of oxygen that human and other animal cells consume when they metabolize food show that anywhere from 25% to 35% of that oxygen is being used to compensate for mitochondrial proton leaks. As such, it was thought that perhaps there were other UCP proteins that account for this uncoupling. In fact, researchers have now identified two additional UCP proteins, UCP-2 and UPC-3. UCP2 is a second, related uncoupling protein that is much more widely expressed in large adult mammals (see, e.g., Fluery, et al, Nature Genetics 15:269-272 (1997) and Tartaglia, et al., PCT Publication No. WO 96/05861, the teachings of both of which are incorporated herein by reference). UCP2 is expressed in a wide range of tissues ranging from the brain to muscle and fat cells. Consistent with a role in the regulation of energy utilization generally, and in diabetes and obesity in particular, the UCP2 gene is upregulated in response to fat feeding and maps to regions of the human and mouse genomes linked to hyperinsulinaemia and obesity. More recently, the UCP3 gene has been characterized and found to be preferentially expressed in skeletal muscle and brown adipose tissues (see, Vidal-Puig, et al. BBRC 235:79-82 (1997) and Boss, et al. FEBS Letters 408:3942 (1997).

Although early evidence suggests that UCP2 behaves like UCP1 and uncouples oxidation and ATP synthesis, there remains a need in the art for compositions and methods that can be used to elucidate the role of UCP2 in the cell and in body weight disorders and to identify modulators of UCP2 polypeptides. The present invention fulfills this and other needs.

### SUMMARY OF THE INVENTION

In one embodiment, the present invention provides isolated and/or recombinant nucleic acids that encode human UCP2 polypeptides. The nucleotide sequences of the human UCP2 nucleic acids of the invention differ from those of previously known UCP2-encoding nucleic acids. More particularly, the UCP2 nucleic acids of the invention encode a UCP2 polypeptide that includes at least 164 consecutive amino acid residues of the amino acid sequence set forth in SEQ ID NO: 1. More particularly, the UCP2 nucleic acids encode a polypeptide that has an alanine at position 55, and a threonine at position 219 of the amino acid sequence set forth in SEQ ID NO: 1. For example, the codons at positions 163-165 of the nucleotide sequence set forth in SEQ ID NO: 2 can be GCT, GCC, GCA or GCG. The codons at positions 655 to 657 of the nucleotide sequence set forth in SEQ ID NO: 2 can be ACT, ACC, ACA or ACG. As mentioned above, the human UCP2 nucleic acids of the invention differ from those described previously. The UCP2 nucleotide sequence described by Fluery, et al., Nature Genetics 15:269-272 (1997), for example, has the codon ATT at positions 655 to 657, whereas the UCP2 nucleotide sequence described by Tartaglia, et al., PCT Publication No. WO 96/05861, has the codon GTC at positions 163-165.

The human UCP2 nucleic acids of the invention find use in many applications. For example, the nucleic acids are useful for producing human UCP2 polypeptides that can be used, for example, in screening assays to identify modulators of UCP2 biological activity, or as pharmaceutical agents to treat body weight disorders, such as obesity, underweight disorders, etc. The UCP2 nucleic acids of the invention are also useful in screening assays to identify compounds that can modulate UCP2 gene expression levels. One can also use the UCP2 nucleic acids of the invention to make antisense and triplex-forming nucleic acids that can inhibit expression of UCP2 genes upon administration to a cell.

In another embodiment, the present invention provides novel isolated human UCP2 polypeptides. The amino acid sequences of the human UCP2 polypeptides of the invention differ from those of previously known UCP2 polypeptides. More particularly, the UCP2 polypeptides of the invention include at least 164 consecutive amino acid residues of the amino acid sequence set forth in SEQ ID NO: 1. Specifically, the UCP2 polypeptides include an alanine at amino acid residue 55 and a threonine at amino acid residue 219 of the amino acid sequence set forth in SEQ ID NO: 1. As such, the human UCP2 polypeptides of the present invention differ from those described by Fluery, et al., Nature Genetics 15:269-272 (1997), which has an isoleucine at position 219, and Tartaglia, et al., PCT Publication No. WO 96/05861, which has a valine at position 55. In a presently preferred embodiment, the human UCP2 polypeptides of the present invention have the amino acid sequence set forth in SEQ ID NO: 1. The polypeptides of the invention also include those in which one or more amino acids at positions other than position 55 and 219 have conservative substitutions.

The UCP2 polypeptides of the present invention find use, for example, in screening assays to identify compounds that can modulate (i.e., increase or decrease) the biological activity of UCP2 polypeptides in a mammal. The UCP2 polypeptides of the invention also are useful for therapeutic use, for example, to treat obese mammals by increasing the rate of fat metabolism.

In other embodiments, the present invention provides recombinant nucleic acid molecules containing nucleic acids encoding a UCP2 polypeptide; cells containing such recombinant nucleic acid molecules; a method for producing UCP2 polypeptides; and methods for detecting modulators of UCP2 gene expression and UCP2 polypeptide expression.

Other features, objects and advantages of the invention and its preferred embodiments will become apparent from the detailed description that follows.

### BRIEF DESCRIPTION OF THE DRAWINGS

FIG. 1 illustrates a comparison of the nucleic acid sequences of the UCP2 nucleic acids identified by Tartaglia, et al., supra (A), Fleury, et al., supra (B) and Chen, SEQ. ID. NO: 2 (C).

FIG. 2 illustrates a comparison of the amino acid sequences of the UCP2 polypeptides identified by Fleury, et al., supra (A), Chen, SEQ. ID. NO: 1 (B) and Tartaglia, et al., supra (C).

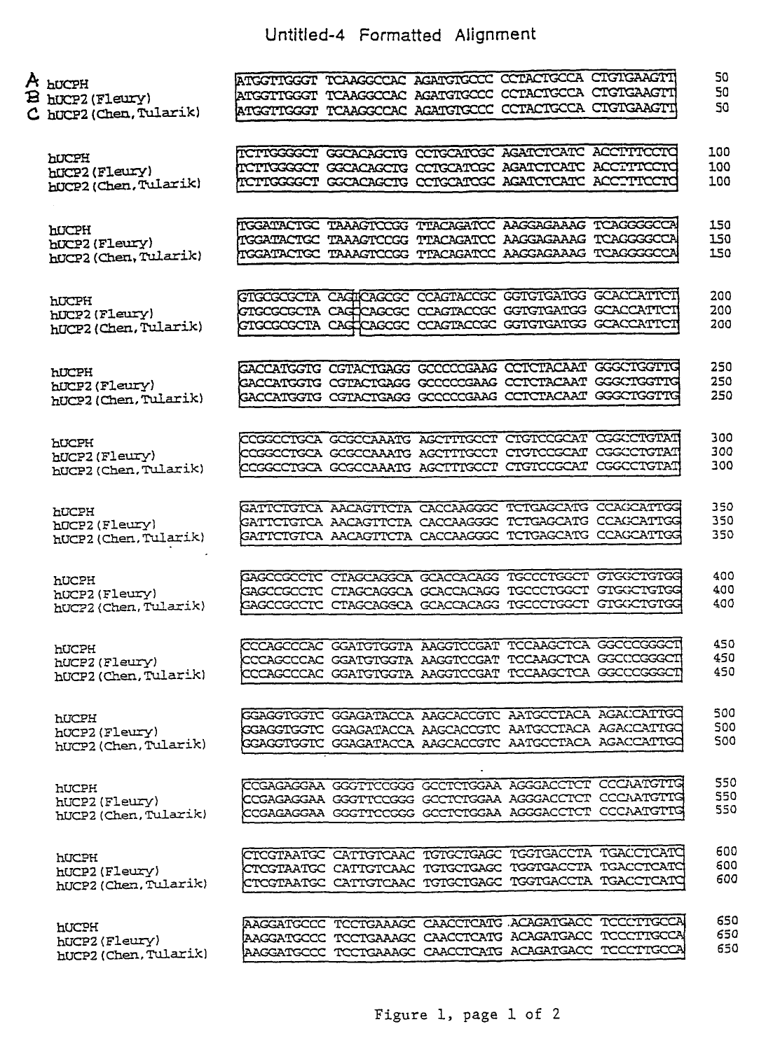

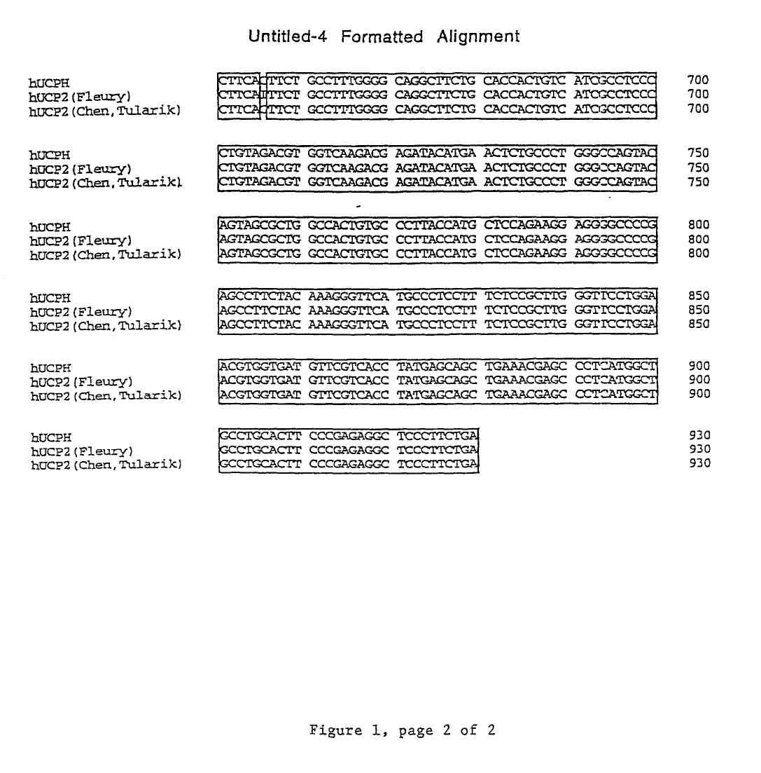

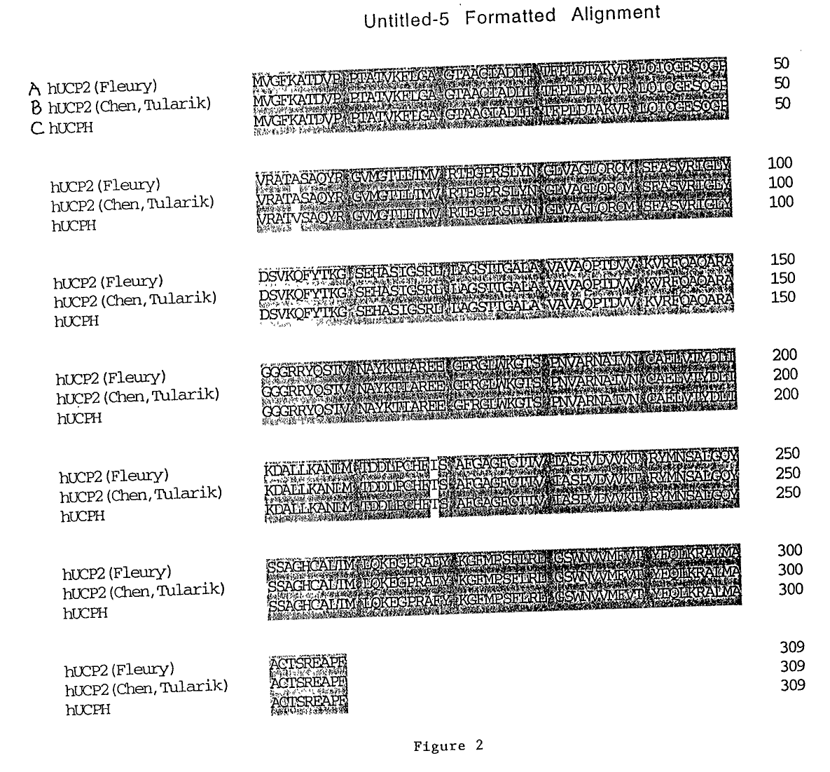

### DETAILED DESCRIPTION OF THE INVENTION AND PREFERRED EMBODIMENTS

A. Definitions

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides, refers to two or more sequences or subsequences that have at least 60%, preferably 80% and, most preferably, 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1994 Supplement) (Ausubel)).

Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated for nucleotide sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions.

Another indication that two nucleic acid sequences are substantially identical is that the two molecules hybridize to each other under stringent conditions. The phrase “hybridizing specifically to,” refers to the binding, duplexing or hybridizing of a molecule only to a particular nucleotide sequence under stringent conditions when that sequence is present in a complex mixture (e.g., total cellular) DNA or RNA. The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Specific hybridization can also occur within a living cell.

The term “nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid that encodes a polypeptide is implicit in each described sequence.

The term “recombinant,” when used with reference to a cell, nucleic acid or vector, indicates that the cell, nucleic acid or vector has been modified by the introduction of a heterologous nucleic acid or the alteration of a native nucleic acid, or that the cell is derived from a cell so modified. Thus, for example, recombinant cells can contain genes that are not found within the native (non-recombinant) form of the cell or can express native genes that are otherwise abnormally expressed, under expressed or not expressed at all. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

B. Human UCP2 Nucleic Acids

Isolated and/or recombinant nucleic acids that encode human UCP2 polypeptides are provided by the present invention. The nucleotide sequences of the human UCP2 nucleic acids of the invention differ from those of previously known UCP2-encoding nucleic acids. The UCP2 nucleic acids of the invention find use in many applications. For example, the nucleic acids are useful for producing human UCP2 polypeptides that can be used, for example, in screening assays to identify modulators of UCP2 biological activity, or as pharmaceutical agents to treat obesity or underweight disorders. The UCP2 nucleic acids of the invention are also useful in screening assays to identify compounds that can modulate UCP2 gene expression levels. One can also use the UCP2 nucleic acids of the invention to make antisense and triplex-forming nucleic acids that can inhibit expression of UCP2 genes upon administration to a cell.

The UCP2 nucleic acids of the invention encode a human UCP2 polypeptide that includes at least 164 consecutive amino acid residues of the amino acid sequence set forth in SEQ ID NO: 1. More particularly, the UCP2 nucleic acids encode a polypeptide that has an alanine at position 55 and a threonine at position 219 of the amino acid sequence set forth in SEQ ID NO: 1. As such, the codons at positions 163-165 of the nucleotide sequence set forth in SEQ ID NO: 2 can be GCT, GCC, GCA or GCG, whereas the codons at positions 655 to 657 of the nucleotide sequence set forth in SEQ ID NO: 2 can be ACT, ACC, ACA or ACG. As such, the human UCP2 nucleic acids of the invention differ from those described previously. The UCP2 nucleotide sequence described by Fluery, et al., Nature Genetics 15:269-272 (1997), for example, has the codon ATT at positions 655 to 657, whereas the UCP2 nucleotide sequence described by Tartaglia, et al., PCT Publication No. WO 96/05861, has the codon GTC at positions 163-165.

In a presently preferred embodiment, the UCP2 nucleic acid of the invention encodes a human UCP2 polypeptide having the amino acid sequence as set forth in SEQ ID NO: 1. One example of a human UCP2 nucleic acid of the invention is that which has the nucleotide sequence as set forth in SEQ ID NO: 2.

The UCP2-encoding nucleic acids, or subsequences (i.e., probes) thereof, of the present invention can be isolated by cloning or amplification using in vitro methods, such as the polymerase chain reaction (PCR), the ligase chain reaction (LCR), the transcription-based amplification system (TAS), the self-sustained sequence replication system (SSR). A wide variety of cloning and in vitro amplification methodologies is well known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

Moreover, examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89: 117.

In one preferred embodiment, UCP2 nucleic acids can be isolated by routine cloning methods. The cDNA sequence provided in SEQ ID NO: 2 can be used to provide probes that specifically hybridize to a UCP2 gene in a genomic DNA sample, to a UCP2 mRNA in a total RNA sample (e.g., in a Southern blot) or to a UCP2 cDNA in a cDNA library. Once the target UCP2 nucleic acid is identified, it can be isolated according to standard methods known to those of skill in the art (see, e.g., Sambrook, Berger, and Ausubel, supra). In another preferred embodiment, the UCP2 nucleic acids of the invention can be isolated by amplification methods, such as polymerase chain reaction (PCR).

The invention also provides nucleic acid constructs in which a UCP2 polynucleotide of the invention is operably linked to a promoter that is functional in a desired host cell. Such constructs are often provided as an “expression cassette,” which can also include other sequences involved in transcription, translation, and posttranslational modification of the UCP2 polypeptide. Examples of suitable promoters and other control sequences are described herein. The invention also provides expression vectors, and host cells that comprise the claimed recombinant nucleic acids.

C. Human UCP2 Polypeptides

The present invention also provides novel isolated human UCP2 polypeptides. The amino acid sequences of the human UCP2 polypeptides of the invention differ from those of previously known UCP2 polypeptides. The human UCP2 polypeptides of the present invention find use, for example, in screening assays to identify compounds that can modulate (i.e., increase or decrease) the biological activity of UCP2 polypeptides in a mammal. The human UCP2 polypeptides of the invention also have numerous therapeutic uses, such as for treating obese mammals by increasing the rate of fat metabolism.

The human UCP2 polypeptides of the invention include at least 164 consecutive amino acid residues of the amino acid sequence set forth in SEQ ID NO: 1. Specifically, the UCP2 polypeptides include an alanine at amino acid residue 55 and a threonine at amino acid residue 219 of the amino acid sequence set forth in SEQ ID NO: 1. The UCP2 polypeptides thus differ from that described by Fluery, et al., Nature Genetics 15:269-272 (1997), which has an isoleucine at position 219, and that described by Tartaglia, et al., PCT Publication No. WO 96/05861, which has a valine at position 55. In a presently preferred embodiment, the UCP2 polypeptides have the amino acid sequence set forth in SEQ ID NO: 1. The polypeptides of the invention also include those in which one or more amino acids at positions other than position 55 and 219 have conservative substitutions.

The human UCP2 polypeptides of the invention can be made by methods known to those of skill in the art. In a preferred embodiment, the UCP2 proteins, or subsequences thereof, are synthesized using recombinant nucleic acid methodologies. Generally, this involves creating a nucleic acid that encodes the polypeptide, modified as desired, placing the nucleic acid in an expression cassette under the control of a particular promoter, expressing the protein in a host, isolating the expressed protein and, if required, renaturing the protein.

The UCP2 polypeptides of the invention can be expressed in a variety of host cells including, but not limited to, E. coli, other bacterial hosts, yeasts, filamentous fungi, and various higher eukaryotic cells, such as the COS, CHO and HeLa cells lines and myeloma cell lines. Techniques for gene expression in microorganisms are described in, for example, Smith, Gene Expression in Recombinant Microorganisms (Bioprocess Technology, Vol. 22), Marcel Dekker, 1994. Examples of useful bacteria include, but are not limited to, Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus. Filamentous fungi that are useful as expression hosts include, but are not limited to, the following genera: Aspergillus, Trichoderna, Neurospora, Penicillium, Cephalosporium, Achlya, Podospora, Mucor, Cochliobolus, and Pyricularia (see, e.g., U.S. Pat. No. 5,679,543 and Stahl and Tudzynski, Eds., Molecular Biology in Filamentous Fungi, John Wiley & Sons, 1992). Synthesis of heterologous proteins in yeast is well known and described in the literature. Methods in Yeast Genetics, Sherman, F., et al., Cold Spring Harbor Laboratory, (1982) is a well recognized work describing the various methods available to produce the UCP2 polypeptides in yeast.

A polynucleotide that encodes a UCP2 polypeptide of the invention can be operably linked to an appropriate expression control sequence for a particular host cell in which the polypeptide is to be expressed. For E. coli, appropriate control sequences include a promoter, such as the T7, trp or lambda promoters, a ribosome binding site and, preferably, a transcription termination signal. For eukaryotic cells, the control sequences typically include a promoter that optionally includes an enhancer derived from immunoglobulin genes, SV40, cytomegalovirus, etc., and a polyadenylation sequence, and may include splice donor and acceptor sequences. In yeast, convenient promoters include GAL1,10 (Johnson and Davies (1984) Mol. Cell. Biol. 4:1440-1448), ADH2 (Russell et al. (1983) J. Biol. Chem. 258:2674-2682), PHO5 (EMBO J. (1982) 6:675-680), and MFα1 (Herskowitz and Oshima (1982) in The Molecular Biology of the Yeast Saccharomyces (eds. Strathem, Jones, and Broach) Cold Spring Harbor Lab., Cold Spring Harbor, N.Y., pp. 181-209).

For expression of the UCP2 polypeptides in multicellular eukaryotes, suitable host cells and promoters are known to those of skill in the art (see, e.g., Cruz and Patterson Tissue Culture (Academic Press, Orlando (1973)); Meth. Enzymology 68 (Academic Press, Orlando, Fla. (1979); Freshney, Culture of Animal Cells: A Manual of Basic Techniques (2d ed., Alan R. Liss, N.Y. (1987)). Useful host cell lines include, but are not limited to, murine myelomas, N51, VERO and HeT cells, SF9 or other insect cell lines, CV-1 and Chinese hamster ovary (CHO) cells. Expression vectors for such cells generally include promoters and control sequences compatible with mammalian cells such as, for example, the commonly used early and late promoters from Simian Virus 40 (SV40), or other viral promoters such as those from polyoma, adenovirus 2, bovine papilloma virus, or avian sarcoma viruses, herpes virus family (such as cytomegalovirus, herpes simplex virus, or Epstein-Barr virus), or immunoglobulin promoters and heat shock promoters (Sambrook, Ausubel, supra.); Meth. Enzymology supra. (1979, 1983, 1987); Pouwells, et al., supra (1987)). In addition, regulated promoters, such as metallothionine (i.e., MT-1 and MT-2), glucocorticoid, or antibiotic gene “switches” can be used. Enhancer regions can also be used in the expression cassettes of the invention.

Expression cassettes are typically introduced into a vector that facilitates entry of the expression cassette into a host cell and maintenance of the expression cassette in the host cell. Vectors that include a polynucleotide that encodes a UCP2 polypeptide are provided by the invention. Such vectors often include an expression cassette that can drive expression of the UCP2 polypeptide. To easily obtain a vector of the invention, one can clone a polynucleotide that encodes the UCP2 polypeptide into a commercially or commonly available vector. A variety of commercially available vectors suitable for use in the present ivention is well known to those of skill in the art. For cloning in bacteria, common vectors include pBR322 derived vectors, such as PBLUESCRIPT™ and λ-phage derived vectors. In yeast, vectors include Yeast Integrating plasmids (e.g., YIp5), Yeast Replicating plasmids (the YRp series plasmids) and pGPD-2. A multicopy plasmid with selective markers, such as Leu-2, URA-3, Trp-1 and His-3, is also commonly used. A number of yeast expression plasmids such as YEp6, YEp13, YEp4 can be used as expression vectors. The above-mentioned plasmids have been fully described in the literature (Botstein, et al. (1979) Gene 8:17-24; Broach, et al. (1979) Gene, 8:121-133). For a discussion of yeast expression plasmids, see, e.g., Parents, B., YEAST (1985), and Ausubel, Sambrook, and Berger, all supra). Expression in mammalian cells can be achieved using a variety of commonly available plasmids, including pSV2, pBC12BI, and p91023, as well as lytic virus vectors (e.g., vaccinia virus, adenovirus, and baculovirus), episomal virus vectors (e.g., bovine papillomavirus), and retroviral vectors (e.g., murine retroviruses).

The nucleic acids that encode the UCP2 polypeptides of the invention can be transferred into the chosen host cell by well-known methods, such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes, among others. Techniques for transforming fingi are well known in the literature and have been described, for instance, by Beggs, Hinnen et al. ((1978) Proc. Natl. Acad. Sci. USA 75: 1929-1933), Yelton et al. ((1984) Proc. Natl. Acad. Sci. USA 81: 1740-1747), and Russell ((1983) Nature 301: 167-169). Procedures for transforming yeast are also well known (see, e.g., Beggs (1978) Nature (London), 275:104-109; and Hinnen et al. (1978) Proc. Natl. Acad. Sci. USA, 75:1929-1933. Transformation and infection methods for mammalian and other cells are described in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel).

Once expressed, the UCP2 proteins can be purified, either partially or substantially to homogeneity, according to standard procedures known to and used by those of skill in the art. Such proceudures include, but are not limited to, ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Once purified, partially or to homogeneity as desired, the polypeptides may then be used (e.g., as therapeutic reagents or as immunogens for antibody production).

Those of skill in the art will recognize that after chemical synthesis, biological expression or purification, the UCP2 protein of the present invention can possess a conformation substantially different from the native conformations of the constituent polypeptides. In this case, it may be necessary to denature and reduce the polypeptide and then to cause the polypeptide to re-fold into the preferred conformation. Methods of reducing and denaturing proteins and inducing re-folding are well known to those of skill in the art (see, Debinski, et al. (1993) J. Biol. Chem., 268: 14065-14070; Kreitman and Pastan (1993) Bioconjug. Chem., 4: 581-585; and Buchner, et al., (1992) Anal. Biochem., 205: 263-270). Debinski, et al., for example, describe the denaturation and reduction of inclusion body proteins in guanidine-DTE. The protein is then refolded in a redox buffer containing oxidized glutathione and L-arginine.

Moreover, those of skill in the art will recognize that modifications can be made to the UCP2 polypeptides without diminishing their biological activity. Some modifications may be made to facilitate the cloning, expression or incorporation of the polypeptide into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

D. Screening Assays For Identifying Compounds that Interact with Human UCP2

Numerous assays can be used to identify compounds that bind to or interact with UCP2, bind to or interact with other cellular proteins that interact with UCP2, and to compounds that interfere with the interaction of UCP2 with other cellular proteins. Such assays are disclosed in PCT Publication No. WO 96/05861, the teachings of which are incorporated herein by reference.

Compounds identified using the assays of the present invention can be useful, for example, in elaborating the biological function of UCP2 and for ameliorating body weight disorders. In instances where a body weight disorder situation results from a lower overall level of UCP2 gene expression, UCP2 polypeptide and/or UCP2 polypeptide activity in a cell or tissue involved in such a body weight disorder, compounds that interact with the UCP2 polypeptide may include ones which accentuate or amplify the activity of the bound UCP2 protein. Such compounds would bring about an effective increase in the level of UCP2 gene activity, thus ameliorating symptoms. In instances where mutations within the UCP2 gene cause aberrant UCP2 proteins to be made which have a deleterious effect that leads to a body weight disorder, compounds that bind UCP2 protein may be identified that inhibit the activity of the bound UCP2 protein.

In vitro systems may be designed to identify compounds capable of binding the UCP2 polypeptides of the invention. Such compounds may include, but are not limited to, peptides made of D-and/or L-configuration amino acids (in, for example, the form of random peptide libraries; see, e.g., Lam, K. S., et. al., 1991, Nature 354:82-84; Houghten, R., et al., 1991, Nature 354:84-86), phosphopeptides (in, for example, the form of random or partially degenerate, directed phosphopeptide libraries; see, e.g., Songyang, Z., et al., 1993, Cell 72:767-778), antibodies, and small organic or inorganic molecules. Compounds identified may be useful, for example, in modulating the activity of UCP2 polypeptides, preferably mutant UCP2 polypeptides, may be useful in elaborating the biological function of the UCP2 polypeptides, may be utilized in screens for identifying compounds that disrupt normal UCP2 polypeptide interactions, or may themselves disrupt such interactions.

The principle of the assays used to identify compounds that bind to the UCP2 polypeptides of the present invention involves preparing a reaction mixture of the UCP2 polypeptide and the test compound under conditions and for a time sufficient to allow the two components to interact and bind, thereby forming a complex which can be removed and/or detected in the reaction mixture. These assays can be conducted in a variety of ways. For example, one method to conduct such an assay involves anchoring the UCP2 polypeptide or the test substance onto a solid phase and detecting UCP2 polypeptide/test compound complexes anchored on the solid phase at the end of the reaction. In one embodiment of such a method, the UCP2 polypeptide may be anchored onto a solid surface, and the test compound, which is not anchored, may be labeled, either directly or indirectly.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, the reaction can be conducted in a liquid phase, the reaction products separated from unreacted components, and complexes detected; e.g., using an immobilized antibody specific for UCP2 polypeptide or the test compound to anchor any complexes formed in solution, and a labeled antibody specific for the other component of the possible complex to detect anchored complexes.

In another embodiment, the UCP2 polypeptides of the invention may, in vivo, interact with one or more cellular or extracellular macromolecules, such as proteins. Such macromolecules may include, but are not limited to, nucleic acid molecules and polypeptides. For purposes of this discussion, such cellular and extracellular macromolecules are referred to herein as “binding partners”. Compounds that disrupt such interactions may be useful in regulating the activity of the UCP2 polypeptide, especially mutant UCP2 polypeptides. Such compounds may include, but are not limited to, molecules such as antibodies, peptides, and the like as described above.

The basic principle of the assay systems used to identify compounds that interfere with the interaction between the UCP2 polypeptide and its cellular or extracellular binding partner or partners involves preparing a reaction mixture containing the UCP2 polypeptide, and the binding partner under conditions and for a time sufficient to allow the two to interact and bind, thereby forming a complex. In order to test a compound for inhibitory activity, the reaction mixture is prepared in the presence and absence of the test compound. The test compound may be initially included in the reaction mixture, or may be added at a time subsequent to the addition of UCP2 polypeptide and its cellular or extracellular binding partner. Control reaction mixtures are incubated without the test compound or with a placebo. The formation of any complexes between the UCP2 polypeptide and the cellular or extracellular binding partner is then detected. The formation of a complex in the control reaction, but not in the reaction mixture containing the test compound, indicates that the compound interferes with the interaction of the UCP2 polypeptide and the interactive binding partner. Additionally, complex formation within reaction mixtures containing the test compound and normal UCP2 polypeptide may also be compared to complex formation within reaction mixtures containing the test compound and a mutant UCP2 polypeptide. This comparison may be important in those cases wherein it is desirable to identify compounds that disrupt interactions of mutant, but not normal UCP2 polypeptides.

The assay for compounds that interfere with the interaction of the UCP2 polypeptides and binding partners can be conducted in a heterogeneous or homogeneous format. Heterogeneous assays involve anchoring either the UCP2 polypeptide or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the UCP2 polypeptides and the binding partners, e.g., by competition, can be identified by conducting the reaction in the presence of the test substance; i.e., by adding the test substance to the reaction mixture prior to or simultaneously with the UCP2 polypeptide and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the UCP2 polypeptide or the interactive cellular or extracellular binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the UCP2 polypeptide or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the UCP2 polypeptide and the interactive cellular or extracellular binding partner is prepared in which either the UCP2 polypeptide or its binding partners is labeled, but the signal generated by the label is quenched due to complex formation (see, e.g., U.S. Pat. No. 4,109,496, which issued to Rubenstein and which utilizes this approach for immunoassays). The addition of a test substance that competes with and displaces one of the species from the preformed complex will result in the generation of a signal above background. In this way, test substances which disrupt UCP2 polypeptide/cellular or extracellular binding partner interaction can be identified.

In other embodiments of the present invention, any of the binding compounds, including but not limited to, compounds such as those identified in the foregoing assay systems, may be tested for the ability to ameliorate body weight disorder symptoms, which may include, for example, obesity, anorexia and/or an abnormal level of food intake. Cell-based and animal model-based assays for the identification of compounds exhibiting such an ability to ameliorate body weight disorder symptoms are described below.

First, cell-based systems, such as those described in PCT Publication No. WO 96/05861, can be used to identify compounds that may act to ameliorate body weight disorder symptoms. For example, such systems may be exposed to a compound suspected of exhibiting an ability to ameliorate body weight disorder symptoms, at a sufficient concentration and for a time sufficient to elicit such an amelioration of body weight disorder symptoms in the exposed cells. After exposure, the cells are examined to determine whether one or more of the body weight disorder-like cellular phenotypes has been altered to resemble a more normal or more wild type, non-body weight disorder phenotype.

In addition, animal-based body weight disorder systems, such as those described in PCT Publication No. WO 96/05861, can be used to identify compounds capable of ameliorating body weight disorder-like symptoms. Such animal models may be used as test substrates for the identification of drugs, pharmaceuticals, therapies and interventions which may be effective in treating such disorders. For example, animal models may be exposed to a compound suspected of exhibiting an ability to ameliorate body weight disorder symptoms, at a sufficient concentration and for a time sufficient to elicit such an amelioration of body weight disorder symptoms in the exposed animals. The response of the animals to the exposure may be monitored by assessing the reversal of disorders associated with body weight disorders such as obesity.

With regard to intervention, any treatments which reverse any aspect of body weight disorder-like symptoms should be considered as candidates for human body weight disorder therapeutic intervention. Dosages of test agents may be determined by deriving dose-response curves using methods known to those of skill in the art In another assay of the invention, test compounds are screened to identify those that can modulate expression of a humanUCP2 gene. A cell is provided that contains a promoter sequence from a UCP2 nucleic acid that is operably linked to a reporter gene. The cell is contacted with a test compound that is a potential modulator of gene expression. Detection of the presence or absence of reporter gene expression is an indicator for whether the test compound is a modulator of UCP2 gene expression. A variety of reporter gene plasmid systems are known, such as the common chloramphenicol acetyltransferase (CAT) and β-galactosidase (e.g., bacterial LacZ gene) reporter systems, the firefly luciferase gene (See, e.g., Cara et al., (1996) J. Biol. Chem., 271: 5393-5397), the green fluorescence protein (see, e.g., Chalfie et al. (1994) Science 263:802) and many others. Selectable markers which facilitate cloning of the vectors of the invention are optionally included. Sambrook and Ausubel, both supra, provide an overview of selectable markers.

E. Methods for Treatment of Body Weight Disorders

In another embodiment, the present invention provides methods and compositions where body weight disorder symptoms may be ameliorated. It is possible that body weight disorders may be brought about, at least in part, by an abnormal level of UCP2 polypeptide, or by the presence of a UCP2 polypeptide exhibiting an abnormal activity. As such, the reduction in the level and/or activity of such UCP2 polypeptides would bring about the amelioration of body weight disorder-like symptoms. Techniques for the reduction of UCP2 gene expression levels or UCP2 polypeptide activity levels are described hereinbelow. Alternatively, it is possible that body weight disorders may be brought about, at least in part, by the absence or reduction of the level of UCP2 gene expression, or a reduction in the level of a UCP2 polypeptide's activity. As such, an increase in the level of UCP2 gene expression and/or the activity of such gene products would bring about the amelioration of body weight disorder-like symptoms. Techniques for increasing UCP2 gene expression levels or UCP2 polypeptide activity levels are also discussed hereinbelow.

More particularly, as discussed above, UCP2 genes involved in body weight disorders may cause such disorders via an increased level of UCP2 gene activity. A variety of techniques may be utilized to inhibit the expression, synthesis, or activity of such UCP2 genes and/or proteins. For example, compounds such as those identified through the assays described above, which exhibit inhibitory activity, may be used in accordance with the invention to ameliorate body weight disorder symptoms. As discussed above, such molecules include, but are not limited to, small organic molecules, peptides, antibodies, and the like. Further, antisense and ribozyme molecules that inhibit expression of the UCP2 gene may also be used in accordance with the invention to inhibit the aberrant UCP2 gene activity. Such antisense and ribozyme molecules and techniques are known to and used by those of skill in the art. Still further, triple helix molecules may be utilized in inhibiting the aberrant UCP2 gene activity. Moreover, antibodies that are both specific for a UCP2 polypeptide and interfere with its activity may be used to inhibit UCP2 gene function. Where desirable, antibodies specific for mutant UCP2 proteins that interfere with the activity of such mutant UCP2 proteins may also be used to inhibit UCP2 gene function. Such antibodies may be generated using standard techniques known to those of skill in the art against the proteins themselves or against peptides corresponding to portions of the proteins. The antibodies include, but are not limited to, polyclonal, monoclonal, Fab fragments, single chain antibodies, chimeric antibodies, etc.

Moreover, as described above, UCP2 genes that cause body weight disorders may be underexpressed within body weight disorder situations. Alternatively, the activity of UCP2 polypeptides may be diminished, leading to the development of body weight disorder symptoms. Those of skill in the art will know of numerous methods whereby the level of UCP2 gene activity may be increased to levels wherein body weight disorder symptoms are ameliorated. For instance, the level of gene activity may be increased, for example, by either increasing the level of UCP2 polypeptide present or by increasing the level of active UCP2 polypeptide which is present.

More particularly, a UCP2 polypeptide, at a level sufficient to ameliorate body weight disorder symptoms, can be administered to a patient exhibiting such symptoms. Any of the techniques discussed below can be utilized for such administration. One of skill in the art will readily know how to determine the concentration of effective, non-toxic doses of the normal UCP2 polypeptide.

Additionally, RNA sequences encoding UCP2 polypeptide may be directly administered to a patient exhibiting body weight disorder symptoms, at a concentration sufficient to produce a level of UCP2 polypeptide such that body weight disorder symptoms are ameliorated. Any of the techniques, which achieve intracellular administration of compounds, such as, for example, liposome administration, may be utilized for the administration of such RNA molecules. The RNA molecules may be produced, for example, by recombinant techniques such as those described above.

Further, patients may be treated by gene replacement therapy. One or more copies of a normal UCP2 gene or a portion of the gene that directs the production of a normal UCP2 polypeptide with UCP2 gene function may be inserted into cells using vectors which include, but are not limited to, adenovirus, adeno-associated virus, and retrovirus vectors, in addition to other particles that introduce DNA into cells, such as liposomes. Additionally, techniques such as those described above may be utilized for the introduction of normal UCP2 gene sequences into human cells.

Cells, preferably, autologous cells, containing normal UCP2 gene expressing sequences may then be introduced or reintroduced into the patient at positions which allow for the amelioration of body weight disorder symptoms. Such cell replacement techniques may be preferred, for example, when the UCP2 polypeptide is a secreted, extracellular gene product.

F. Pharmaceutical Compositions of the Human UCP2 Polypeptides and Human UCP2 Nucleic Acids

The human UCP2 polypeptides and nucleic acids of the invention find use in preventing and treating weight gain disorders in humans and other mammals. Accordingly, the present invention provides pharmaceutical compositions that contain a UCP2 polypeptide or nucleic acid dissolved or dispersed in a pharmaceutically acceptable carrier or diluent. In therapeutic applications, a composition is administered to a patient already suffering from a condition associated with metabolic disorders that affect body weight, as described above, in an amount sufficient to inhibit or enhance fat metabolism as is appropriate for the particular condition; i.e., to cure or at least partially arrest the symptoms of the condition and its complications. An amount adequate to accomplish this is defined as a “therapeutically effective dose” or an “effective amount.” As will be seen from the following disclosure, an effective amount can vary. That amount is, however, generally sufficient to inhibit or enhance UCP2 biological activity in a cell by about 2% or more and, more preferably, by about 10% or more.

Amounts effective for this use depend on the severity of the condition and the weight and general state of the patient, but generally range from about 0.5 mg to about 10,000 mg of UCP2 polypeptide or nucleic acid per day for a 70 kg patient, with dosages of from about 5 mg to about 2,000 mg of a compound per day being more commonly used. To formulate a range of therapeutically effective doses for humans, one can use data obtained from cell culture assays and animal studies. For example, one can determine the ED₅₀ of a compound using cell culture assays, and then use a dose that provides a circulating plasma concentration range that is at least as high as the ED₅₀. The dosage can vary within this range depending upon the dosage form employed and the route of administration utilized. Levels in plasma may be measured, for example, by high performance liquid chromatography.

The dose of the compound varies according to, e.g., the particular UCP2 polypeptide or nucleic acid, the manner of administration, the particular body weight disorder being treated and its severity, the overall health and condition of the patient, and the judgment of the prescribing physician. Ideally, therapeutic administration should begin as soon as possible after the disorder is discovered. Successful treatment using a contemplated pharmaceutical composition can be determined by the state of development of the condition to be treated.

In prophylactic applications, a composition containing a contemplated compound is administered to a patient susceptible to or otherwise at risk of a particular disorder. An amount of compound sufficient to obtain prophylaxis is defined to be a “prophylactically effective dose” and is also an amount sufficient to inhibit or enhance weight gain, as desired. In this use, the precise amounts again depend on the patient's state of health and weight, but generally range from about 0.5 mg to about 5,000 mg per 70 kilogram patient and, more commonly, from about 5 mg to about 2,000 mg per 70 kg of body weight.

Single or multiple administrations of a composition can be carried out with dose levels and patterns being selected by the treating physician. In any event, the pharmaceutical formulations should provide a quantity of a UCP2 polypeptide or nucleic acid sufficient to effectively treat the patient.

A contemplated pharmaceutical composition is comprised of a human UCP2 polypeptide or human UCP2 nucleic acid of the present invention, which compound is dissolved or dispersed in a pharmaceutically acceptable diluent. A contemplated pharmaceutical composition is suitable for use in a variety of drug delivery systems. Suitable formulations for use in the pharmaceutical compositions of the present invention are found in, for example, Remington's Pharmaceutical Sciences, Mace Publishing Company, Philadelphia, Pa., 17th ed. (1985). For a brief review of methods for drug delivery, see, Langer (1990) Science 249: 1527-1533.

A pharmaceutical composition is intended for parenteral, topical, oral or local administration, such as by aerosol or transdermally, for prophylactic and/or therapeutic treatment. A pharmaceutical composition can be administered in a variety of unit dosage forms depending upon the method of administration. For example, unit dosage forms suitable for oral administration include powder, tablets, pills, capsules and dragees.

Preferably, a pharmaceutical composition is administered intravenously. Thus, this invention provides a composition for intravenous administration that comprises a solution of a contemplated UCP2 compound dissolved or dispersed in a pharmaceutically acceptable diluent (carrier), preferably an aqueous carrier. A variety of aqueous carriers can be used, e.g., water, buffered water, 0.4 percent saline, and the like. The compositions may contain pharmaceutically acceptable auxiliary substances as required to approximate physiological conditions, such as pH adjusting and buffering agents, tonicity adjusting agents, wetting agents, detergents and the like, for example, sodium acetate, sodium lactate, sodium chloride, potassium chloride, calcium chloride, sorbitan monolaurate, triethanolamine oleate, etc.

These compositions can be sterilized by conventional, well known sterilization techniques, or can be sterile filtered. The resulting aqueous solutions can be packaged for use as is, or lyophilized, the lyophilized preparation being combined with a sterile aqueous solution prior to administration. The pH of the preparations typically will be between 3 and 11, more preferably from 5 to 9 and most preferably from 7 and 8.

The concentration of UCP2 polypeptide or nucleic acid utilized is usually at or at least about 1 percent to as much as 10 to 30 percent by weight and is selected primarily by fluid volumes, viscosities, etc., in accordance with the particular mode of administration selected. Thus, a typical pharmaceutical composition for intravenous infusion can be made up to contain 250 ml of sterile Ringer's solution, and 25 mg of the UCP2 polypeptide. Actual methods for preparing parenterally administrable compounds are known or apparent to those skilled in the art and are described in more detail in, for example, Remington's Pharmaceutical Sciences, 17th ed., Mack Publishing Company, Easton, Pa. (1985). The UCP2 polypeptides and UCP2 nucleic acids of the present invention can also be delivered via liposome preparations.

For solid compositions, conventional nontoxic solid diluents (carriers) may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95 percent of active ingredient, that is, a UCP2 polypeptide or UCP2 nucleic acid of the present invention, preferably about 20 percent (see, Remington's, supra.).

For aerosol administration, a contemplated UCP2 polypeptide or nucleic acid compound is preferably supplied in finely divided form along with a surfactant and propellant. Typical percentages of a UCP2 compound are about 0.5 to about 30 percent by weight, and preferably about 1 to about 10 percent by weight. The surfactant must, of course, be nontoxic and, preferably, soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride such as, for example, ethylene glycol, glycerol, erythritol, arabitol, mannitol, sorbitol, the hexitol anhydrides derived from sorbitol, and the polyoxyethylene and polyoxypropylene derivatives of these esters. Mixed esters, such as mixed or natural glycerides can be employed. The surfactant can constitute about 0.1 to about 20 percent by weight of the composition, and preferably about 0.25 to about 5 percent by weight. The balance of the composition is ordinarily propellant. Liquefied propellants are typically gases at ambient conditions, and are condensed under pressure. Among suitable liquefied propellants are the lower alkanes containing up to 5 carbons, such as butane and propane and, preferably, fluorinated or fluorochlorinated alkanes. Mixtures of the above can also be employed. In producing the aerosol, a container equipped with a suitable valve is filled with the appropriate propellant, containing the finely divided compounds and surfactant. The ingredients are thus maintained at an elevated pressure until released by action of the valve.

The invention will be described in greater detail by way of specific examples. The following examples are offered for illustrative purposes, and are intended neither to limit nor define the invention in any manner.

### EXAMPLES

The following example describes a procedure for cloning a human UCP2 cDNA.

A cDNA library prepared from human fat cells was subjected to PCR amplification using the primers U1F (5′-ATCAAGCTTATGGTTGGGTTCAAGGCCACAGATG-3′; SEQ ID NO: 3) and U8R (5′-ATCGGATCCTCAGAAGGGAGCCTCTCGGGAAGC-3′, SEQ ID NO: 4). The U1F primer includes a HindIII restriction site (underlined), and the U8R primer includes a BamHI restriction site (underlined). Primers were diluted to 10 μM in water for use as stock solutions.

The PCR reaction mixtures were as follows:

| Ingredient | Volume | |
|---|---|---|
| Human fat cell cDNA | 1 | μl |
| Forward primer (U1F), 10 μM stock | 1 | μl |
| Reverse primer (U8R), 10 μM stock | 1 | μl |
| dNTPs, 10 mM total (2.5 mM each) stock | 1 | μl |
| 10X Taq Buffer | 5 | μl |
| MgCl₂, 25 mM stock | 2 | μl |
| ddH₂O | 34 | μl |
| TOTAL | 45 | μl |

All of the reaction components, except for Taq buffer and Taq polymerase, were heated to 94° C. for 3 minutes and cooled to 80° C. for 5 minutes, at which time the Taq buffer (5 μl) and Taq polymerase (1 μl, 5 units) were added. The reaction mixture was then subjected to 35 PCR cycles of 94° C. for 1 minute, 57° C. for 2 minutes, and 72° C. for 2 minutes. The reaction mixture was then incubated at 72° C. for 10 minutes, and finally incubated at 4° C. for up to 24 hours.

A fragment of approximately 1 kb in length was amplified and cloned into a Bluescript vector at the HindIII and BamHI sites. Sequencing of this fragment and analysis of the deduced amino acid sequence resulted in the discovery that the UCP2 polynucleotide sequence described herein has an alanine at amino acid residue 55 and a threonine at amino acid residue 219.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. An isolated UCP2 polypeptide, said UCP2 polypeptide comprising at least 164 consecutive amino acid residues of the amino acid sequence set forth in SEQ. ID. NO: 1, said consecutive amino acid residues comprising an alanine at amino acid residue 55 and a threonine at amino acid residue 219 of SEQ. ID. NO: 1.

2. The isolated UCP2 polypeptide in accordance with claim 1, wherein said UCP2 polypeptide has the amino acid sequence set forth in SEQ. ID. NO: 1.

3. The isolated UCP2 polypeptide in accordance with claim 1, wherein said UCP2 polypeptide is encoded by the nucleic acid sequence set forth in SEQ. ID. NO: 2.

4. An isolated nucleic acid that encodes a UCP2 polypeptide, wherein the codon for amino acid residue 55 (Ala) is a member selected from the group consisting of GCT, GCC, GCA and GCG, and the codon for amino acid residue 219 (Thr) is a member selected from the group consisting of ACT, ACC, ACA and ACG.

5. The isolated nucleic acid that encodes a UCP2 polypeptide in accordance with claim 4, wherein said codon for amino acid residue 55 is GCC.

6. The isolated nucleic acid that encodes a UCP2 polypeptide in accordance with claim 4, wherein said codon for amino acid residue 219 is ACT.

7. The isolated nucleic acid that encodes a UCP2 polypeptide in accordance with claim 4, wherein said UCP2 polypeptide has the amino acid sequence set forth in SEQ. ID. NO: 1.

8. The isolated nucleic acid that encodes a UCP2 polypeptide in accordance with claim 4, wherein said nucleic acid has the nucleic acid sequence set forth in SEQ. ID. NO: 2.

9. An isolated nucleic acid that encodes the UCP2 polypeptide of claim 1, wherein a codon for amino acid residue 55 (Ala) is a member selected from the group consisting of GCT, GCC, GCA and GCG, and a codon for amino acid residue 219 (Thr) is a member selected from the group consisting of ACT, ACC, ACA and ACG.

10. An isolated nucleic acid that encodes a UCP2 polypeptide in accordance with claim 4, wherein said nucleic acid is operably linked to a promoter.

11. An isolated nucleic acid that encodes a UCP2 polypeptide in accordance with claim 10, wherein said nucleic acid is contained in an expression vector.

12. An expression vector containing the nucleic acid of claim 4 in operative association with a regulatory element that controls expression of the nucleic acid in a host cell.

13. A cell comprising a recombinant nucleic acid in accordance with claim 4.

14. A cell in accordance with claim 13, wherein said recombinant nucleic acid is in operative association with a regulatory element that controls the expression of the nucleic acid in a host cell.

15. A method of making a UCP2 polypeptide, said method comprising:
introducing a nucleic acid of claim 4 into a host cell or cellular extract;
incubating said host cell or cellular extract under conditions such that said UCP2 polypeptide is expressed in said host cell or cellular extract; and
recovering said UCP2 polypeptide from said host cell or cellular extract.

16. A method for diagnosing body weight disorders, said method comprising detecting in a patient sample, the level of:
a. an mRNA transcribed from a nucleic acid encoding a UCP2 polypeptide having the amino acid sequence set forth in SEQ. ID. NO: 1;
b. a UCP2 polypeptide having the amino acid sequence set forth in SEQ.ID.NO:1;or
c. a UCP2 polypeptide encoded by the nucleic acid sequence set forth in SEQ. ID. NO: 2.

17. The method in accordance with claim 16, wherein the level is induced in overweight individuals.

18. The method in accordance with claim 16, wherein the level is repressed in overweight individuals.

19. The method in accordance with claim 16, wherein the level is induced in underweight individuals.

20. The method in accordance with claim 16, wherein the level is repressed in underweight individuals.

21. A method of treating obesity in a mammal, said method comprising administering to said mammal a therapeutically effective amount of a UCP2 polypeptide and a pharmaceutically acceptable carrier.

22. A method of identifying a modulator of UCP2 gene expression, said method comprising:
providing a cell comprising a UCP2 promoter operably linked to a reporter gene;
contacting said cell with a test compound that is a potential modulator of UCP2 gene expression; and
detecting the expression level of the reporter gene, wherein an increase or decrease in reporter gene expression in the presence of the test compound compared to reporter gene expression in the absence of the test compound indicates that the test compound is a modulator of UCP2 gene expression.

23. The method according to claim 22, wherein the test compound causes an increase in reporter gene expression.
